(19) United States
(12) Patent Application Publication
(10) Pub. No.: US 2025/0387638 A1
(43) Pub. Date: Dec. 25, 2025

(54) Phototherapy Helmet

(71) Applicant: Shenzhen Kaiyan Medical Equipment Co., Ltd., Shenzhen (CN)

(72) Inventors: Alain Dijkstra, Amstelveen (NL); Li XIANG, Shenzhen (CN); Liu Junlin, Shenzhen (CN); Fan Lin, Shenzhen (CN); Li Quan, Shenzhen (CN); Guo Chunfang, Shenzhen (CN); Wen Caiyun, Shenzhen (CN); Zhou Hong, Shenzhen (CN); Chen Xin, Shenzhen (CN)

(73) Assignee: Shenzhen Kaiyan Medical Equipment Co., Ltd., Shenzhen (CN)

(21) Appl. No.: 19/238,583

(22) Filed: Jun. 16, 2025

(30) Foreign Application Priority Data
Jun. 19, 2024 (CN) 2024107978555
Jun. 19, 2024 (CN) 2024214179529
Jun. 26, 2024 (CN) 2024108351955
Jun. 26, 2024 (CN) 2024214760339
Dec. 16, 2024 (CN) 2024231112962

(51) Int. Cl. A61N 5/06; A61N 5/00

(52) U.S. Cl. CPC A61N 5/06; A61N 2005/005; A61N 2005/0632; A61N 2005/0647; A61N 2005/0652

(57) ABSTRACT

A flexible light therapy helmet is disclosed. The flexible light therapy helmet comprises an outer layer formed of Ethylene Vinyl Acetate (EVA) material via an injection molding process. The outer layer defines a wearing space. An inner layer formed of the EVA material via the injection molding process. The inner layer is disposed on a side of the outer layer facing the wearing space. A printed circuit board (PCB) is disposed between the inner layer and the outer layer. The PCB comprises a plurality of light-emitting diodes (LEDs). A plurality of light-transmitting holes formed in the inner layer. Each light-transmitting hole is aligned with a corresponding LED to transmit therapeutic light toward a user's scalp.

### TECHNICAL FIELD

This disclosure relates generally to the field of personal care devices and more particularly to a flexible light therapy helmet and method of manufacturing the same.

### BACKGROUND

Light-based therapy has gained widespread adoption for treating various dermatological, cosmetic, and medical conditions. Among these applications, phototherapy for the scalp, particularly for promoting hair growth and stimulating acupuncture points has proven to be both effective and non-invasive. Devices using light-emitting diodes (LEDs) are commonly employed to emit red, blue, or infrared light, which is known to stimulate blood circulation, promote cellular regeneration, and enhance overall scalp health.

Traditional phototherapy devices often take the form of rigid helmets or cap-like structures that house the light sources and require external power sources and are constructed using metal components which result in an overweight and cumbersome design. These configurations tend to be bulky, uncomfortable to wear for long periods, and difficult to manufacture. Moreover, their rigid form factors do not conform well to varying head shapes, leading to uneven light exposure and reducing therapeutic efficacy. Another limitation of conventional designs is the absence of multi-modal therapeutic features. While light therapy is effective on its own, combining it with secondary stimulation such as vibration, electrical pulses, or thermal elements can significantly enhance the treatment outcome. However, integrating such features into a comfortable, flexible, and mass-producible device remains a significant technical challenge. In simple terms, current light therapy caps are uncomfortable, difficult to manufacture, and not very adaptable to different head shapes. They often miss out on the chance to combine light therapy with other treatments like gentle massage or electrical stimulation, which could make the treatment more effective. People using these devices may feel heat buildup, limited contact, or poor light coverage which reduces their comfort and the benefits they get.

Therefore, there is a need for a light therapy helmet that is flexible, lightweight, easy to wear, and integrally manufactured using a streamlined injection molding process. Such a device should allow for the embedded placement of light sources while providing modular or integrated elements for additional therapeutic stimulation.

### SUMMARY OF THE INVENTION

In an embodiment, a flexible light therapy helmet is disclosed. The flexible light therapy helmet may include an outer layer formed of Ethylene Vinyl Acetate (EVA) material via an injection molding process. In an embodiment, the outer layer defines a wearing space. The flexible light therapy helmet may further include an inner layer formed of the EVA material via the injection molding process. In an embodiment, the inner layer may be disposed on a side of the outer layer facing the wearing space. The flexible light therapy helmet may further include a printed circuit board (PCB) disposed between the inner layer and the outer layer. In an embodiment, the PCB may include a plurality of light-emitting diodes (LEDs). The flexible light therapy helmet may further include a plurality of light-transmitting holes formed in the inner layer. In an embodiment, each light-transmitting hole is aligned with a corresponding LED to transmit therapeutic light toward a user's scalp.

In accordance with the embodiment, the inner layer and the outer layer may be fixed together via glue or via the injection molding process.

In accordance with the embodiment, each light-transmitting hole may include a plug-in part covering the LED and extending outward from the inner layer. In an embodiment, the plug-in part may include a lens configured to enhance the light emitted by the LED.

In accordance with the embodiment, the plug-in part may be coupled to an elastic pad. In an embodiment, the elastic pad may include an elastic belt and a plug-in receiving hole for receiving the corresponding plug-in part.

In accordance with the embodiment, the plug-in part may be integrally formed with the elastic pad, and the elastic pad may be affixed to the inner layer.

In accordance with the embodiment, the elastic pad may include a conductive pathway to supply electrical power to a stimulation element.

In another embodiment, a flexible light therapy helmet is disclosed. The flexible light therapy helmet may include an outer layer formed of Ethylene Vinyl Acetate (EVA) material via an injection molding process. In an embodiment, the outer layer defines a wearing space. The flexible light therapy helmet may further include an inner layer formed of the EVA material via the injection molding process. In an embodiment, the inner layer may be disposed on a side of the outer layer facing the wearing space. The flexible light therapy helmet may further include a plurality of plug-in parts disposed within a plurality of light-transmitting holes aligned with the LEDs. The flexible light therapy helmet may further include a plurality of elastic protrusions coupled to the plug-in parts, each elastic protrusion comprising a secondary stimulation element configured to deliver one or more electrical, vibrational, or thermal stimulation to a user scalp.

In accordance with the embodiment, the inner layer and the outer layer may be fixed together via glue or via the injection molding process.

In accordance with the embodiment, the secondary stimulation element may be selected from a group of an electrode, a vibration motor, and a thermal module.

In accordance with the embodiment, each elastic protrusion may include a silicon tip and a metal contact surface configured to contact the user's scalp.

In accordance with the embodiment, the stimulation element is electrically connected to the PCB via an embedded wire or conductive trace.

In accordance with the embodiment, the plug-in part may include an angular groove having a first limiting surface and a second limiting surface configured to abut opposite sides of the inner layer.

In accordance with the embodiment, the elastic protrusion may include a sleeve portion housing the stimulation element and a buffer portion to absorb mechanical pressure.

In accordance with the embodiment, the plug-in parts and elastic protrusions are integrally formed with the elastic pad.

In accordance with the embodiment, the flexible light therapy helmet may further include a decorative strip disposed on an external surface of the outer layer.

In accordance with the embodiment, the PCB may be mounted on a flexible substrate and may include arc-shaped light strips arranged in longitudinal and traverse directions.

In yet another embodiment, a method for manufacturing a flexible light therapy helmet is disclosed. The method may include positioning a printed circuit board (PCB) having a plurality of light-emitting diodes (LEDs) between an inner mold and an outer mold. The method may further include injecting ethylene-vinyl acetate (EVA) material into the inner mold and the outer mold to form an inner layer and an outer layer such that the PCB is embedded between the inner layer and the outer layer. The method may further include forming a plurality of light-transmitting holes in the inner layer, wherein each light-transmitting hole is aligned with a corresponding LED.

In accordance with the embodiment, the method may further include inserting a plurality of plug-in parts into the respective light-transmitting holes, each plug-in part disposed to cover the corresponding LED.

In accordance with the embodiment, the method may further include attaching a plurality of elastic protrusions to the respective plug-in parts. In an embodiment, each protrusion may include a stimulation element configured to provide one or more electrical vibrational, or thermal stimulations to a user.

In accordance with the embodiment, each stimulation element may be electrically connected to the PCB via one of a direct electrical lead embedded during molding, or a conductive trace disposed within an elastic pad coupled to the elastic protrusion.

In accordance with the embodiment, forming the plurality of light-transmitting holes may include positioning a plurality of core pins in the mold aligned with the LEDs before injection of the EVA material.

It is to be understood that both the foregoing general description and the following detailed descriptions are exemplary and explanatory only and are not restrictive of the invention, as claimed.

### OBJECTIVES OF THE INVENTION

An objective of the present invention is to provide a flexible light therapy helmet that is integrally formed through an injection molding process, thereby simplifying the manufacturing workflow and improving product consistency. By embedding a printed circuit board (PCB) with light-emitting diodes (LEDs) between inner and outer layers made of Ethylene Vinyl Acetate (EVA), the invention achieves a lightweight, durable, and flexible structure capable of delivering therapeutic light to a user's scalp.

Another objective of the present invention is to enable precise light delivery by aligning each LED with a corresponding light-transmitting hole formed in the inner EVA layer. This alignment ensures that therapeutic light is directed effectively toward the user's scalp without substantial diffusion or loss.

A further objective of the invention is to enhance the functionality of the light therapy helmet by incorporating plug-in parts and elastic protrusions capable of housing secondary stimulation elements, such as electrodes, vibration modules, or thermal components. These additional therapeutic modalities improve the efficacy and customization of treatment options for various dermatological or wellness applications.

Yet another objective of the present invention is to simplify assembly and improve mechanical integrity by forming the plug-in parts, elastic pads, and elastic protrusions as a single molded component, optionally integrated during the same injection molding cycle as the EVA layers. This one-go molding approach reduces part count, minimizes assembly errors, and enables scalable, cost-efficient production.

### SUMMARY OF THE INVENTION

According to a first aspect of the present invention, a flexible light therapy helmet is provided. The flexible light therapy helmet comprising: an outer layer formed of Ethylene Vinyl Acetate (EVA) material, wherein the outer layer defines a wearing space; an inner layer formed of the EVA material, wherein the inner layer is disposed on a side of the outer layer facing the wearing space; a printed circuit board (PCB) disposed between the inner layer and the outer layer, wherein the PCB comprising a plurality of light-emitting diodes (LEDs); and a plurality of light-transmitting holes formed in the inner layer, wherein each light-transmitting hole aligned with a corresponding LED to transmit therapeutic light toward a user scalp.

In one embodiment of the invention, the inner layer and the outer layer are fixed together via glue or via the injection molding process.

In one embodiment of the invention, the inner layer and the outer layer are formed by an injection molding process.

In one embodiment of the invention, each light-transmitting hole comprises a plug-in part covering the LED and extending outward from the inner layer, wherein the plug-in part comprises a lens configured to enhance the light emitted by the LED.

In one embodiment of the invention, the plug-in part is coupled to an elastic pad, wherein the elastic pad comprising an elastic belt and a plug-in receiving hole for receiving the corresponding plug-in part.

In one embodiment of the invention, the plug-in part is integrally formed with the elastic pad, and the elastic pad is affixed to the inner layer.

In one embodiment of the invention, the elastic pad comprises a conductive pathway to supply electrical power to a stimulation element.

In one embodiment of the invention, the inner layer and the outer layer comprise one or more air vents.

According to a second aspect of the present invention, a flexible light therapy helmet is provided. The flexible light therapy helmet comprising: an outer layer formed of Ethylene Vinyl Acetate (EVA) material via an injection molding process, wherein the outer layer defining a wearing space; an inner layer formed of the EVA material, wherein the inner layer is disposed on a side of the outer layer facing the wearing space, and wherein the inner layer is joined with the outer layer; a printer circuit board (PCB) disposed between the inner layer and the outer layer, wherein the PCB comprising a plurality of light-emitting diodes (LEDs); a plurality of light-transmitting holes formed in the inner layer, wherein each light-transmitting hole aligned with a corresponding LED to transmit therapeutic light toward a user scalp; a plurality of plug-in parts each disposed within a corresponding light-transmitting hole; and a plurality of elastic protrusions coupled to the plug-in parts, each elastic protrusion comprising a secondary stimulation element configured to deliver one or more of electrical, vibrational, or thermal stimulation to a user scalp.

In one embodiment of the invention, the secondary stimulation element is selected from a group consisting of: an electrode, a vibration motor, and a thermal module.

In one embodiment of the invention, each elastic protrusion comprises a silicon tip and a metal contact surface configured to contact the user's scalp.

In one embodiment of the invention, the plug-in part comprises an angular groove having a first limiting surface and a second limiting surface configured to abut opposite sides of the inner layer.

In one embodiment of the invention, the elastic protrusion comprises a sleeve portion housing the stimulation element and a buffer portion to absorb mechanical pressure.

In one embodiment of the invention, the plug-in parts and elastic protrusions are integrally formed with the elastic pad.

In one embodiment of the invention, the flexible light therapy helmet further comprises a decorative strip disposed on an external surface of the outer layer.

In one embodiment of the invention, the PCB is mounted on a flexible substrate and comprises arc-shaped light strips arranged in longitudinal and transverse directions.

According to a third aspect of the present invention, a method for manufacturing a flexible light therapy helmet is provided. The method comprising: positioning a printed circuit board (PCB) having a plurality of light-emitting diodes (LEDs) between an inner mold and an outer mold; injecting ethylene-vinyl acetate (EVA) material into the inner mold and the outer mold to form an inner layer and an outer layer such that the PCB is embedded between the inner layer and the outer layer; and forming a plurality of light-transmitting holes in the inner layer, wherein each light-transmitting hole is aligned with a corresponding LED.

In one embodiment of the invention, the method further comprising: inserting a plurality of plug-in parts into the respective light-transmitting holes, each plug-in part disposed to cover the corresponding LED.

In one embodiment of the invention, the method further comprising: attaching a plurality of elastic protrusions to the respective plug-in parts, wherein each elastic protrusion comprising a stimulation element configured to provide one or more of electrical, vibrational, or thermal stimulation to a user.

In one embodiment of the invention, the method further comprising: forming the plurality of light-transmitting holes comprises positioning a plurality of core pins in the mold aligned with the LEDs before injection of the EVA material.

In the context of this specification, terms such as “outer layer” and “inner layer” refer to two structurally distinct components of the helmet body, both preferably formed of Ethylene Vinyl Acetate (EVA) material. The outer layer is positioned on the exterior side of the helmet, facing away from the user's scalp, while the inner layer is positioned adjacent to the user's head, defining the wearing space. These layers may be formed concurrently or sequentially during an injection molding process.

The term “printed circuit board (PCB)” refers to a flexible or semi-rigid substrate on which a plurality of light-emitting diodes (LEDs) are mounted. The PCB may include conductive pathways, connectors, and optional interface components necessary for operating the LEDs and any additional stimulation elements. The PCB is embedded between the outer layer and inner layer during the molding process to form an integrated electronic and mechanical structure.

As used herein, “light-transmitting holes” refer to through-holes, cavities, or channels formed in the inner layer, each being spatially aligned with an underlying LED. These holes allow light generated by the LEDs to be directed toward the user's scalp. In some embodiments, each light-transmitting hole may include or be enclosed by a “plug-in part,” which may function as a lens, housing, or attachment point for additional components such as elastic protrusions or elastic pads.

The term “elastic protrusion” refers to a deformable, typically silicone-based, extension coupled to the plug-in part. It may include a cavity or sleeve configured to house a “secondary stimulation element,” such as an electrode, vibration motor, or thermal module. These stimulation elements are configured to deliver additional therapeutic modalities to the user's scalp and may be electrically connected to the PCB via embedded wires or conductive traces.

“Elastic pad” as used in this disclosure refers to a structure made of flexible material that conforms to the scalp-facing surface of the inner layer. It may support or house multiple elastic protrusions and may include “elastic belts” or “plug-in receiving holes” for mechanical coupling with the plug-in parts. The pad may be formed integrally with the protrusions or affixed separately using Velcro, magnetic coupling, or adhesive methods.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first’, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “flexible substrate” refers to a base material that supports the placement and interconnection of electrical components, while allowing the overall structure to bend or conform to non-planar surfaces. Examples include polyimide film, thermoplastic elastomers, and silicone-based sheets.

In the context of the specification, the term “helmet body” or “wearable housing” refers to a structure configured to be worn on the head or another body part, designed to house and retain functional components such as light sources, sensors, and control electronics. The housing may be rigid, semi-rigid, or flexible, and may be formed from materials such as polycarbonate, EVA foam, or silicone rubber.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

### BRIEF DESCRIPTION OF THE DRAWING

The accompanying drawings, which are incorporated in and constitute a part of this disclosure, illustrate exemplary embodiments and, together with the description, serve to explain the disclosed principles.

To more clearly illustrate the technical solutions described in the embodiments of the present invention, the accompanying drawings referenced below are provided as part of the detailed description. It should be understood that these drawings represent only certain exemplary embodiments of the invention, and that other configurations and variations may be derived from these illustrations by those skilled in the art without departing from the scope of the present disclosure.

FIG. 1 illustrates a first perspective view of a flexible light therapy helmet, in accordance with an embodiment of the present disclosure.

FIG. 2 illustrates an exploded view of the flexible light therapy helmet of FIG. 1, in accordance with an embodiment of the present disclosure.

FIG. 3 illustrates a sectional view of the flexible light therapy helmet of FIG. 1, in accordance with an embodiment of the present disclosure.

FIG. 4 illustrates a cross-sectional view along line AA in FIG. 3, in accordance with an embodiment of the present disclosure.

FIG. 5 illustrates a partially enlarged view of point B in FIG. 4, in accordance with an embodiment of the present disclosure.

FIG. 6 illustrates a second perspective view of the flexible light therapy helmet, in accordance with an embodiment of the present disclosure.

FIG. 7 illustrates a third perspective view of the flexible light therapy helmet, in accordance with an embodiment of the present disclosure.

FIG. 8 illustrates a fourth perspective view of the flexible light therapy helmet, in accordance with an embodiment of the present disclosure.

FIG. 9 illustrates a fifth perspective view of the flexible light therapy helmet, in accordance with an embodiment of the present disclosure.

FIG. 10 illustrates an exploded view of the flexible light therapy helmet, in accordance with an embodiment of the present disclosure.

FIG. 11 illustrates a cross-sectional view of the flexible light therapy helmet, in accordance with an embodiment of the present disclosure.

FIG. 12 illustrates an enlarged view of point A in FIG. 11, in accordance with an embodiment of the present disclosure.

FIG. 13 illustrates a perspective view of elastic protrusion, plug-in portion, and elastic pad in FIG. 9, in accordance with an embodiment of the present disclosure.

FIG. 14 illustrates a perspective view of the elastic protrusion, plug-in portion, and elastic pad in FIG. 13 at another angle, in accordance with an embodiment of the present disclosure.

FIG. 15 illustrates an enlarged view of point B in FIG. 14, in accordance with an embodiment of the present disclosure.

FIG. 16 illustrates a perspective view of an inner layer in FIG. 10, in accordance with an embodiment of the present disclosure.

FIG. 17 illustrates a plan view of the inner layer in FIG. 16, in accordance with an embodiment of the present disclosure.

FIG. 18 illustrates a perspective view of an outer layer in FIG. 10, in accordance with an embodiment of the present disclosure.

FIG. 19 illustrates an enlarged view of point C in FIG. 11, in accordance with an embodiment of the present disclosure.

FIG. 20 illustrates a perspective view of a flexible circuit board in FIG. 10, in accordance with an embodiment of the present disclosure.

FIG. 21 is a flowchart of a methodology for manufacturing the flexible light therapy helmet, in accordance with an embodiment of the present disclosure.

### DETAILED DESCRIPTION OF THE DRAWINGS

The foregoing description has broadly outlined the features and technical advantages of the present disclosure in order that the detailed description of the disclosure that follows may be better understood. Additional features and advantages of the disclosure will be described hereinafter which forms the subject of the claims of the disclosure. It should be appreciated by those skilled in the art that the conception and specific embodiments disclosed may be readily utilized as a basis for modifying other devices, systems, assemblies, and mechanisms for conducting the same purposes of the present disclosure. It should also be realized by those skilled in the art that such equivalent constructions do not depart from the scope of the disclosure as set forth in the appended claims. The novel features that are believed to be characteristics of the disclosure, to its device or system, together with further objects and advantages will be better understood from the following description when considered in connection with the accompanying figures. It is to be expressly understood, however, that each of the figures is provided for the purpose of illustration and description only and is not intended as a definition of the limits of the present disclosure.

The technical solutions described in the embodiments of the present disclosure will be clearly and fully explained below with reference to the accompanying drawings. It should be understood that the embodiments provided herein are merely illustrative and do not represent the entirety of possible implementations. Variations and modifications to these embodiments that may be conceived by those skilled in the art, without departing from the spirit and scope of the invention and without the exercise of inventive faculty, are intended to fall within the scope of protection afforded by the present disclosure.

It should be noted that, in the description of the present invention, the terms “first,” “second,” and the like are used solely for the purpose of distinguishing between different elements and should not be construed as indicating any order, priority, or relative importance. Such terms are not intended to imply a specific sequence or quantity unless explicitly stated. Accordingly, references to “first” and “second” components may each refer to one or more of such components. Furthermore, the term “and/or” as used herein is intended to encompass any and all combinations of one or more of the associated listed items. For example, the expression “A and/or B” should be interpreted to include: A alone, B alone, or both A and B together.

The terms “including”, “comprises”, “comprising”, “comprising of” or any other variations thereof, are intended to cover a non-exclusive inclusion, such that a system or a device that comprises a list of components or steps does not include only those components or steps but may include other components or steps not expressly listed or inherent to such setup or device. In other words, one or more elements in a system or apparatus proceeded by “comprises . . . a” does not, without more constraints, preclude the existence of other elements or additional elements in the system or apparatus.

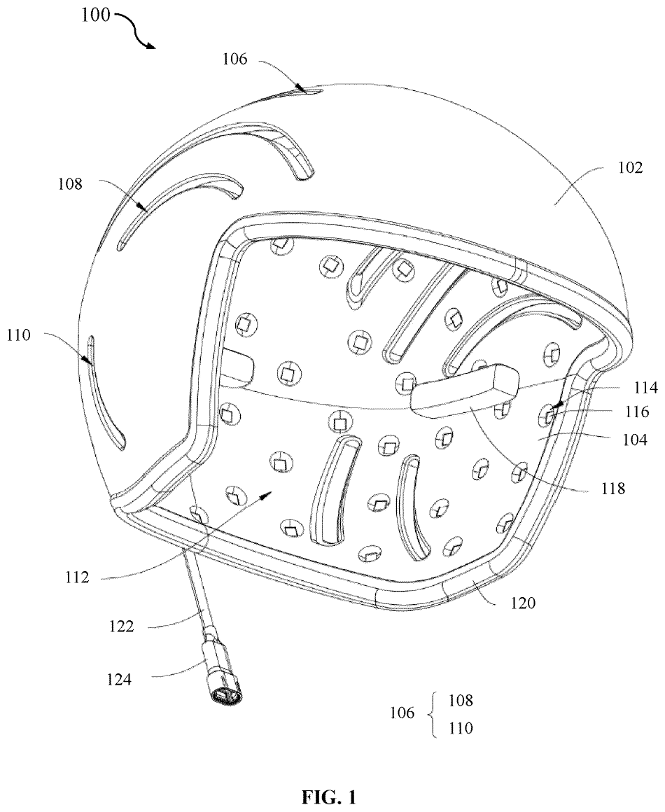

Reference will now be made to the exemplary embodiments of the disclosure, as illustrated in the accompanying drawings. Wherever possible, the same numerals have been used to refer to the same or like parts. The following paragraphs describe the present disclosure with reference to FIGS. 1-21. As summarized above, in one broad aspect, the present invention provides a flexible light therapy helmet and a method of manufacturing the same.

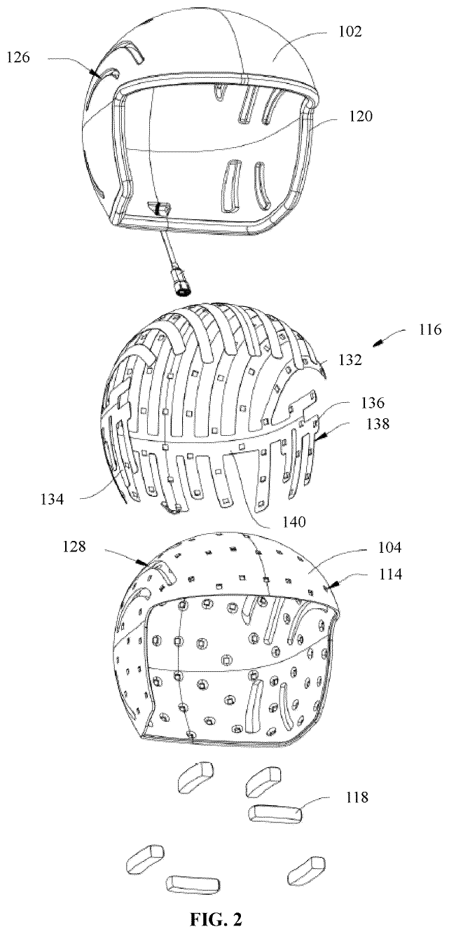

Referring to FIG. 1 and FIG. 2, a flexible light therapy helmet 100 is illustrated, in accordance with an embodiment of the present disclosure. The flexible light therapy helmet 100 comprises an outer layer 102 and an inner layer 104, both formed of Ethylene Vinyl Acetate (EVA) material. In an alternate embodiment, the flexible light therapy helmet 100 is constructed using a non-EVA structural material, such as a metallic or semi-rigid composite material. In this configuration, the flexible light therapy helmet 100 comprises the outer layer 102 and the inner layer 104 made of thermally conductive materials, such as aluminum alloy or stainless steel, which facilitate both mechanical support and passive heat dissipation during light therapy sessions. The outer layer 102 defines a wearing space 112 configured to receive a user's scalp. The inner layer 104 is disposed on a side of the outer layer 102 facing the wearing space 112 and is joined with the outer layer 102 via glue or an injection molding process, thereby forming an integrally molded helmet structure.

In some embodiments, the process of joining the outer layer 102 and the inner layer 104 involves the sequential molding of the EVA layers and subsequently bonding of the inner layer 104 to the outer layer 102. Initially, the outer layer 102 is formed in a dedicated mold cavity using EVA injection molding techniques. Separately, the inner layer 104 is then molded to conform to the curvature of the outer layer 102, with dimensions suitable to define the scalp-facing surface of the flexible light therapy helmet 100.

After molding, the inner layer 104 is fixed to the outer layer 102 using one of: a thermal bonding process, glue, or via a co-injection molding method wherein the outer layer 102 is molded first, and then the inner layer 104 is formed by injecting EVA onto the previously formed outer layer 102 within the same or a corresponding mold. Alternatively, in other embodiments, the inner layer 104 is separately molded and then attached to the outer layer 102 via adhesive bonding techniques. For instance, a layer of industrial adhesive, such as 3M pressure-sensitive bonding tape or EVA-compatible thermal glue, is applied to one or both bonding surfaces. The two layers are then compressed and fixed together, followed by post-curing under heat and pressure to ensure structural integrity.

The bonding interface further includes stitched or sewn edges and is reinforced via ultrasonic welding or over-molding techniques to improve durability, particularly at the edge regions of the flexible light therapy helmet 100. In some embodiments, the inner layer 104 includes alignment features, such as positioning ribs or bonding grooves, to facilitate accurate alignment with the outer layer 102 during the joining process.

In some embodiments, the flexible light therapy helmet 100 is prepared in a single stretch. The flexible light therapy helmet 100 is laminated with a high-elasticity four-way stretch fabric. This fabric enables the molded EVA structure to conform to a user's head while maintaining integrity during the forming process. The fabric is bonded to the surface of the EVA material using a heat-activated adhesive or pressure-sensitive glue. The laminated sheet comprises the EVA substrate and the bonded fabric layer is then shaped in a dedicated helmet mold. During molding, the high-elastic fabric undergoes uniform stretching to match the contours of the mold without wrinkling or tearing.

A flexible light board 116 is embedded between the outer layer 102 and the inner layer 104 during the injection molding process. The flexible light board 116 includes a printed circuit board (PCB) 138 formed of a flexible printed circuit (FPC) material, which enables it to conform to the curved contour of the flexible light therapy helmet 100. The PCB 138 comprises a plurality of light-emitting diodes (LEDs) 136 to deliver therapeutic light. The flexible light board 116 can further include one or more connecting light strips 140, a plurality of first light strips 132, and a plurality of second light strips 134 arranged in arcuate patterns across the surface to optimize light distribution.

In an embodiment, the outer layer 102 and the inner layer 104 are prepared separately from ethylene-vinyl acetate (EVA) material through injection molding processes. After preparation of the outer layer 102 and the inner layer 104, the PCB 138 is affixed to the inner surface of the inner layer 104 using a double-sided adhesive. Thereafter, the outer layer 102 and the inner layer 104 are sewn together or laminated to form a unified layered structure with the flexible light board 116 embedded in between.

A plurality of light-transmitting holes 114 are formed in the inner layer 104. Each light-transmitting hole 114 is aligned with a corresponding LED 136 on the flexible light board 116 to direct therapeutic light through the inner layer 104 and onto the user's scalp. In an embodiment, each light-transmitting hole 114 includes a locking groove to receive and secure a plug-in part. The locking groove provides a mechanical interface that prevents axial or lateral displacement of the plug-in part.

In the illustrated embodiment, the flexible light therapy helmet 100 further includes a flip part 120 formed along the opening edge of the outer layer 102. The flip part 120 extends toward the wearing space 112 and may overlap a peripheral portion of the inner layer 104 to enhance structural stability and improve comfort during wear.

A set of cushion blocks 118 is positioned on the surface of the inner layer 104 facing the wearing space 112. The set of cushion blocks 118 provides mechanical support and user comfort and is removably affixed via glue, magnetic attachment, or co-molded as an integral feature of the inner layer 104. The flexible light therapy helmet 100 further includes a wire 122 and a charging port 124. The wire 122 extends from the PCB 138 and connects to the charging port 124, which is mounted on or passes through the outer layer 102. The charging port 124 is configured to interface with external sources and supply electrical energy to the embedded LEDs, either directly or via an internal rechargeable battery.

A plurality of air vents 106 are formed in the flexible light therapy helmet 100 to enhance breathability. The plurality of air vents 106 comprises one or more first air vents 108 and one or more second air vents 110 positioned symmetrically on opposing sides of the outer layer 102. The plurality of air vents 106 are oriented to follow the curvature of the flexible light therapy helmet 100 to support airflow and heat dissipation during operation. In an embodiment, a metal part can be attached to one or more of the air vents 106. The metal part provides an aesthetically appealing decorative element while also functioning as a passive heat sink to absorb and dissipate heat generated during therapy.

FIG. 2 illustrates an exploded view of the flexible light therapy helmet 100 showing a spatial configuration of its components. The outer layer 102 is provided with a first ventilation sub-hole 126 formed through its surface. The flexible light board 116 includes the plurality of first light strips 132 and the plurality of second light strips 134 and the one or more connecting light strips 140 are positioned between the outer layer 102 and the inner layer 104. The PCB 138 and the LEDs 136 are clearly visible on the flexible light board 116. The inner layer 104 is provided with the plurality of light-transmitting holes 114 and second ventilation sub-holes 128 below the flexible light board 116. The set of cushion blocks 118 is attachable to the interior surface of the inner layer 104 for user comfort.

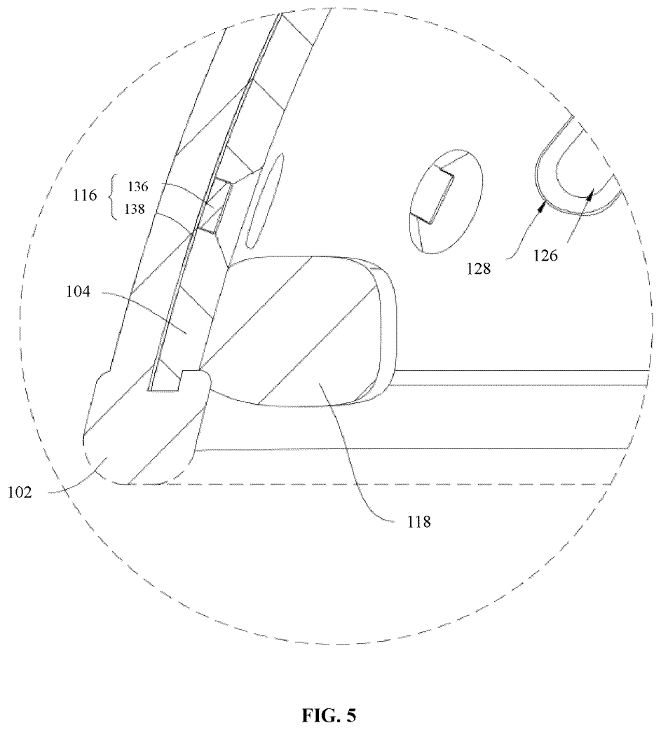

Referring to FIGS. 2 and 5, in some embodiments of the present disclosure, the flexible light board 116 includes the PCB 138 and the plurality of LEDs 136 mounted thereon. The PCB 138 is positioned between the outer layer 102 and the inner layer 104 and conforms to the curved surface of the flexible light therapy helmet 100, providing a flexible and integrable electronic interface for the plurality of LEDs 136. In an embodiment, the outer layer 102 and the inner layer 104 are separately formed from ethylene-vinyl acetate (EVA) material through respective injection molding processes. Once formed, the PCB 138 is affixed to the inner surface of the inner layer 104 using a double-sided adhesive. Thereafter, the outer layer 102 and the inner layer 104 are sewn together or laminated to form a unified layered structure with the flexible light board 116 embedded in between. Each LED 136 is disposed to face the inner layer 104, and aligned with a corresponding light-transmitting hole 114 formed in the inner layer 104.

In some embodiments, each LED 136 is surface-mounted or soldered onto the PCB 138. Upon energization via an external power source, the LEDs 136 are surface-mounted or soldered onto the PCB 138. Upon energization via an external power source, the LEDs 136 emit therapeutic light, which passes through the plurality of light-transmitting holes 114 directed toward the user's scalp.

The plurality of light-transmitting holes 114 are positioned in the inner layer 104 at locations corresponding to the underlying LEDs 136, thereby allowing the emitted light to exit the inner layer 104 with minimal obstruction. The plurality of light-transmitting holes 114 are designed conically tapered or cylindrical and may include plug-in parts to optimize light focusing or control thermal contact.

In one embodiment, the charging port 124 is used to supply electrical power to the PCB 138 via the wire 122. The circuit logic on the PCB 138 controls the actuation and modulation of the LEDs 136, enabling automated or programmatic control over the therapeutic lighting treatment. This configuration simplifies the power delivery mechanism and supports both wired and battery-based operation of the flexible light therapy helmet 100.

The use of the plurality of light-transmitting holes 114 within the inner layer 104 also enables design flexibility. The inner layer 104 is made of a light-transmitting or non-light transmitting or non-light-transmitting material depending on manufacturing constraints, user requirements, or cost considerations. The formation of the plurality of light-transmitting holes 114 at specific locations allows precise control of light paths while maintaining structural integrity, thereby reducing material usage and facilitating case of molding.

Referring to FIGS. 2 and 5, the flexibility light therapy helmet 100 also includes the plurality of air vents 106 configured to enhance breathability and user comfort. Each air vent 106 includes a first air vent 108 formed through the outer layer 102 and a corresponding second air vent 110 formed through the inner layer 104. These paired vents are aligned in the thickness direction of the flexible light therapy helmet 100, forming continuous pathways that connect the wearing space 112 with the external environment.

The plurality of air vents 106 are strategically distributed across the flexible light therapy helmet 100 to promote thermal dissipation and reduce internal heat accumulation during operation. This feature is particularly important when using multiple LEDs 136, which generate heat during prolonged use. The inclusion of first air vents 108 and second air vents 110 allows for improved airflow and ventilation, thereby reducing the sensation of heat and stuffiness for the user.

In some embodiments, the plurality of air vents 106 also reduces the overall material density and weight of the flexible light therapy helmet 100, thus improving comfort during extended wear. Furthermore, the plurality of air vents 106 enhances the flexibility of the outer layer 102 and the inner layer 104 by reducing bulk, facilitating elastic deformation, and easier donning and removal of the flexible light therapy helmet 100.

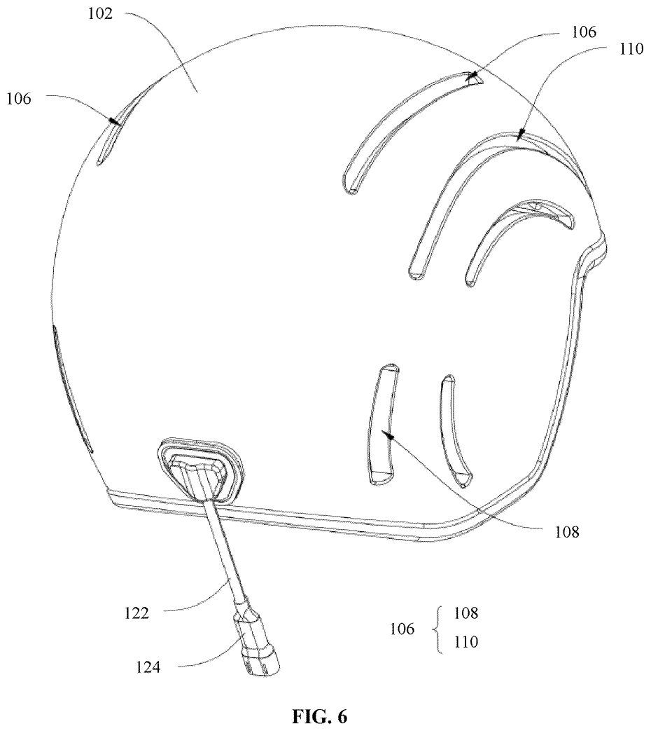

Referring to FIG. 2 in conjunction with FIGS. 5 and 6, the flexible light board 116 includes the PCB 138, the one or more connecting light strips 140, the plurality of first light strips 132, and the plurality of second light strips 134. The PCB 138 comprises multiple electrically conducive tracks and support structures to enable power distribution across the flexible light board 116.

The one or more connecting light strips 140 extend in an arc-shaped manner in a lateral direction, i.e., from left to right across the flexible light therapy helmet 100. The plurality of first light strips 132 extend in an arc from a front region to a back region of the flexible light therapy helmet 100 and are spaced at regular intervals from left to right. The rear end of each of the plurality of first light strips 132 is coupled to the one or more connecting light strips 140. The plurality of second light strips 134 extends in an arc-shaped fashion from a top region toward a bottom region of the flexible light therapy helmet 100. The plurality of second light strips 134 are spaced apart from left to right and have their respective upper ends coupled to the one or more connecting light strips 140. The LEDs 136 are mounted on the surfaces of the plurality of first light strips 132 and the plurality of second light strips 134 and face towards the wearing space 112 to emit therapeutic light toward the user's scalp.

Each LED 136 is configured to be aligned with a corresponding light-transmitting hole 114 formed in the inner layer 104. The plurality of light-transmitting holes 114 allows light emitted from the LEDs 136 to be transmitted with minimal obstruction to the scalp of the user. In some embodiments, the plurality of air vents 106 includes the first air vents 108 and the second air vents 110. The first air vent 108 is defined between two adjacent first light strips 132, and the second air vent 110 is defined between two adjacent second light strips 134. These vents pass through both the outer layer 102 and the inner layer 104 and may be arc-shaped in structure.

The plurality of air vents 106 extends in an arc from the front region (e.g., forehead) to the back region (e.g., nape) of the user's head, conforming to the natural curvature of the head. In some examples, the plurality of air vents 106 are distributed along the left and right sides of the flexible light therapy helmet 100, particularly near the regions corresponding to the user's ears, to provide improved thermal dissipation and ventilation.

The placement of the first air vents 108 and second air vents 110 in the spaces between the plurality of first light strips 132 and the plurality of second light strips 134 forms a distributed pattern of hollow areas that enhance airflow. This arrangement also prevents obstruction of the flexible light board 116, as the one or more connecting light strips 140 maintain uninterrupted power delivery across all connected light strips. The hollow regions formed between the light strips contribute to a reduction in overall weight and material usage of the flexible light board 116. This structural configuration simplifies the design and allows for the integration of the charging port 124 and the wire 122 for supplying electrical power to the LEDs 136.

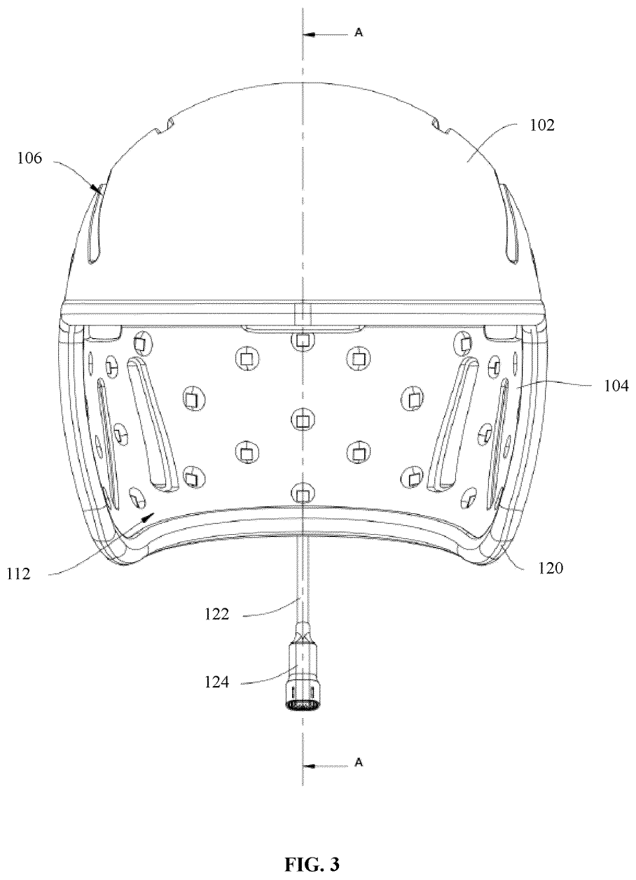

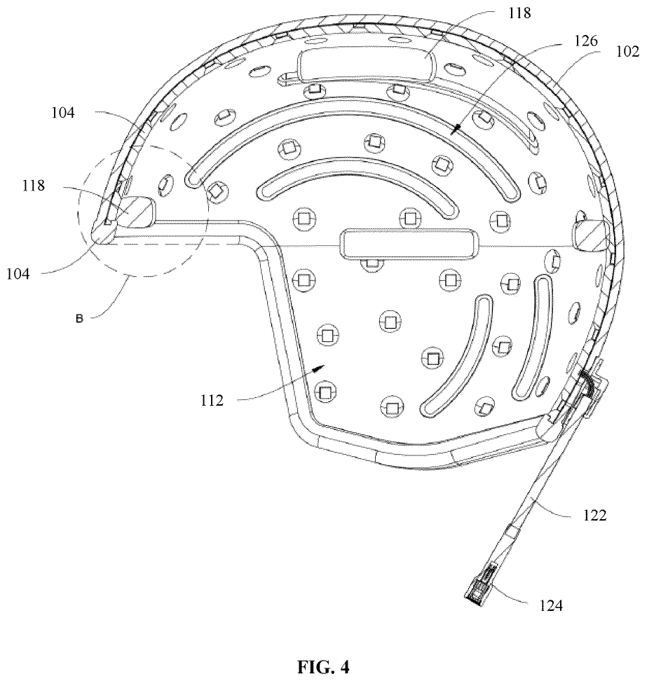

Referring now to FIGS. 3 and 4, the flexible light therapy helmet 100 includes the outer layer 102 formed of Ethylene Vinyl Acetate (EVA) material, and the inner layer 104 also formed of EVA material, both integrally joined via an injection molding process. The space enclosed between the inner layer 104 and outer 102 defines a wearing space 112 configured to receive a user's head.

The plurality of light-transmitting holes 114 are provided in the inner layer 104 and are spatially aligned with respective light-emitting diodes (LEDs) 136 mounted on the PCB 138. The PCB 138, together with the LEDs 136 forms the flexible light board 116. The flexible light board 116 is embedded between the inner layer 104 and the outer layer 102 during the injection molding process. The plurality of first light strips 132 and the plurality of second light strips 134 are arranged longitudinally and transversely on the flexible light board 116 and are interconnected by the one or more connecting light strips 140. The arrangement of the flexible light board 116 ensures even distribution of the therapeutic light toward the user's scalp through the plurality of light-transmitting holes 114. The wire 122 is connected to the flexible light board 116 and extends outward through the outer layer 102. The charging port 124 is connected to a distal end of the wire 122 and is configured to receive external power input, for example, through a USB or Type-C connector, to supply electrical energy to the embedded PCB 138 and LEDs 136.

The set of cushion blocks 118 is disposed on the surface of the inner layer 104 facing the wearing space 112. Each cushion block 118 is configured to protrude inward toward the user's scalp and is made of soft, flexible material. The set of cushion blocks 118 is integrally formed with the inner layer 104 during molding, or affixed post-molding via glue. As illustrated in the sectional view of FIG. 4, the set of cushion blocks 118 increases the gap between the LEDs 136 and the user's scalp, thereby optimizing the uniformity of light exposure and enhancing comfort by enabling airflow through the wearing space 112.

Referring to FIG. 5, an enlarged view of point B of FIG. 4 is illustrated, highlighting the interface between the corresponding cushion block 118 and the inner layer 104. As shown, the corresponding cushion block 118 is molded as an integral extension of the inner layer 104 or mechanically bonded post-molding. Additionally, ventilation features such as the first ventilation sub-hole 126 and the second ventilation sub-hole 128 are disposed of adjacent to or around the corresponding cushion block 118 to facilitate heat dissipation and air exchange during use.

Referring now to FIG. 6, the flexible light therapy helmet 100 includes a plurality of air vents 106, each composed of the first air vent 108 and the second air vent 110. The plurality of air vents 106 can be arc-shaped and are distributed from the frontal region toward the rear region of the outer layer 102 in a manner aligned with the user's scalp anatomy. This arrangement enhances airflow through the flexible light therapy helmet 100, reduces thermal buildup during operation, and improves overall comfort. The wire 122 and the charging port 124 may be again depicted extending through a recess in the outer layer 102. The wire 122 may be flexibly positioned to accommodate user motion and minimize strain at the connection point.

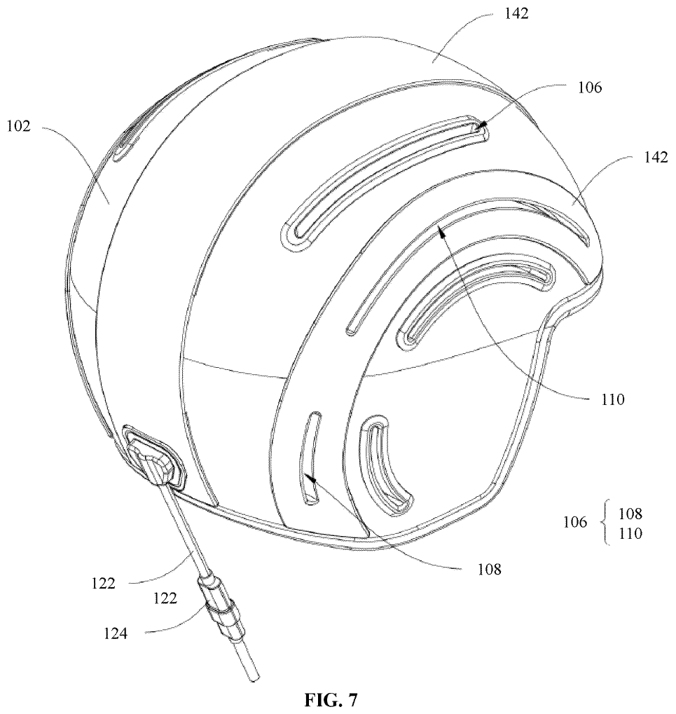

Referring to FIG. 7, a decorative strip 142 is shown disposed along the outer surface of the outer layer 102. At least a portion of the decorative strip 142 is located entirely on the external surface of the outer layer 102 facing away from the wearing space 112, while other portions are embedded into the outer layer 102 or extend partially toward the inner layer 104. The decorative strip 142 improves the aesthetics of the flexible light therapy helmet 100 and contributes to its structural stability. The decorative strip 142 is symmetrically arranged from left to right on the outer layer 102 to enhance both functionality and appearance.

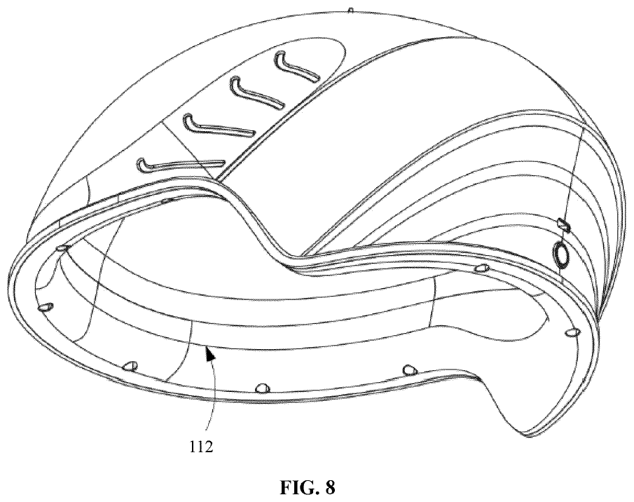

Referring now to FIG. 8, a partial perspective view of the flexible light therapy helmet 100, is illustrated, in accordance with an embodiment of the present disclosure. As shown, the flexible light therapy helmet 100 includes the outer layer 102 formed of a flexible material such as ethylene-vinyl acetate (EVA) and shaped to accommodate a user's head. The outer layer 102 defines a concave inner region referred to as the wearing space 112, configured to receive the user's scalp during wear.

The wearing space 112 is bounded circumferentially by the inner surface of the outer layer 102 and is adapted to receive the inner layer 104 that comprises the PCB 138 and the plurality of light-transmitting holes 114. A plurality of insertion holes 150 are distributed along the boundary of the wearing space 112. Each insertion hole 150 is configured to receive a corresponding plug-in part which extends toward the interior of the flexible light therapy helmet 100 to interface with other internal structures such as elastic protrusions or secondary stimulation elements as described with reference to FIGS. 1 through 7. The secondary stimulation elements include but are not limited to a micro-current electrode, a vibration element, a heating element, a cooling element, or a combination thereof. The micro-current electrode aids in providing micro-current therapy to the scalp of the user in addition to the phototherapy. The vibration element provides massage therapy to the user, and the heating element and cooling element are generally used to provide heat therapy and cold therapy respectively.

In some embodiments, the plurality of insertion holes 150 are generally formed during the injection molding process of the outer layer 102 or can be machined afterward, and are dimensioned to support mechanical engagement with plug-in components that may be either integrally molded or independently installed. The placement of the insertion holes 150 along the wearing space 112 ensures that functional modules, including plug-in parts, magnetic coupling components, or elastic pads, are modularly integrated without interfering with the structural integrity of the flexible light therapy helmet 100.

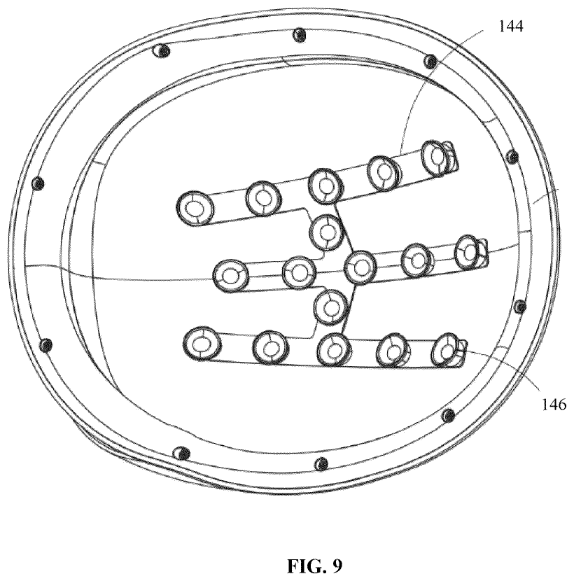

Referring to FIG. 9, an elastic pad 144 and a plurality of elastic protrusions 146 are illustrated. The clastic pad 144 is configured to be installed along a portion of the wearing space 112 of the flexible light therapy helmet 100 and conforms to the curvature of the inner layer 104. The elastic pad 144 can include a support platform for mounting the plurality of clastic protrusions 146, which extend downwardly into the wearing space 112. Each elastic protrusion 146 is configured to house a secondary stimulation element and engages with a corresponding insertion hole 150 formed in the inner layer 104. The secondary stimulation element includes but is not limited to a micro-current electrode, a vibration element, a heating element, a cooling element, or a combination thereof. In this view, the plurality of clastic protrusions 146 are arranged in a geometrically distributed pattern that ensures uniform contact across the scalp when the flexible light therapy helmet 100 is worn, thereby enhancing therapeutic coverage and comfort.

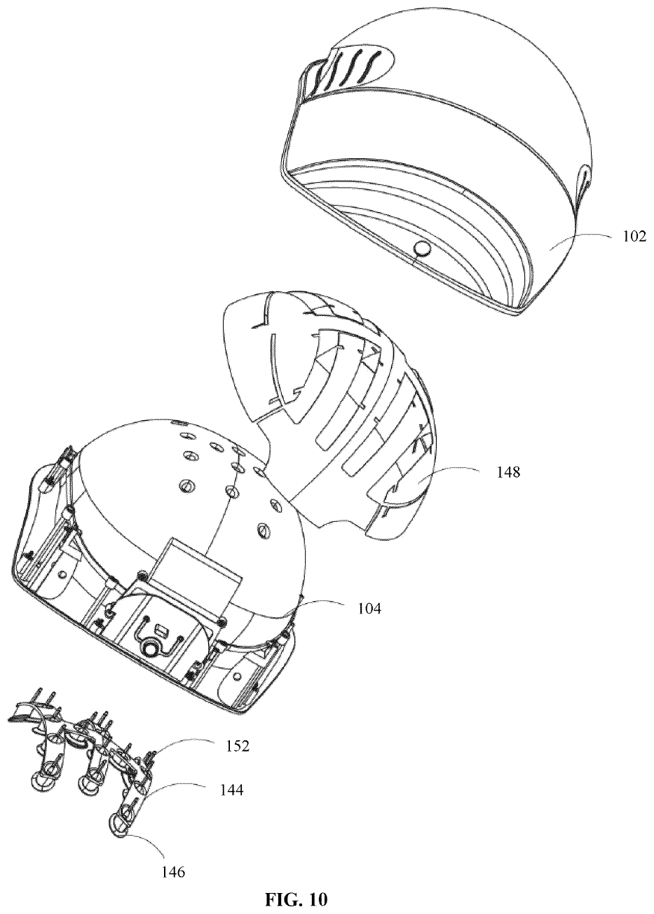

Referring to FIG. 10, an exploded view of the flexible light therapy helmet 100 is shown. The flexible light therapy helmet 100 includes the outer layer 102, the inner layer 104, and a phototherapy lamp 148 configured to be disposed between them. The phototherapy lamp 148 generally includes a flexible light-emitting structure that conforms to the shape of the flexible light therapy helmet 100. The elastic pad 144 is shown separately from the inner layer 104 and is configured to receive the plurality of clastic protrusions 146. A plurality of plug-in parts 152 are also shown, each configured to mechanically secure a corresponding elastic protrusion 146 to the inner layer 104 through the corresponding insertion hole 150.

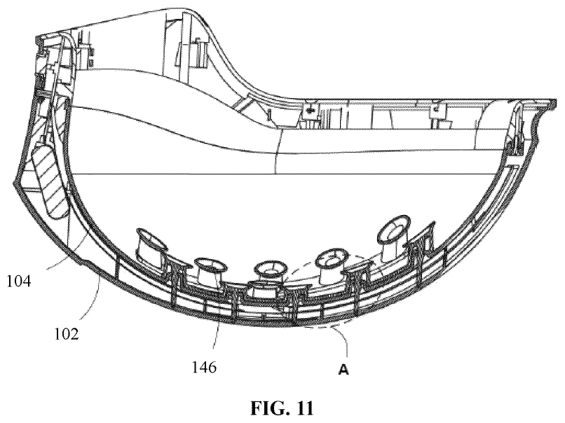

Referring to FIG. 11, a sectional view of the flexible light therapy helmet 100 taken along lines A-A in FIG. 3 is illustrated. This figure clearly shows the embedded configuration of the plurality of elastic protrusions 146 within the inner layer 104 and the outer layer 102. Each elastic protrusion 146 is oriented to project inwardly into the wearing space 112. The clastic pad 144 provides a mounting base for each of the plurality of elastic protrusions 146 and maintains their spatial alignment. The sectional view further demonstrates the integration of the phototherapy lamp 148 between the inner layer 104 and the outer layer 102, as well as the thickness profiles of both layers. The plurality of elastic protrusions 146 are configured to deliver therapeutic stimulation and to ensure effective contact with the user's scalp.

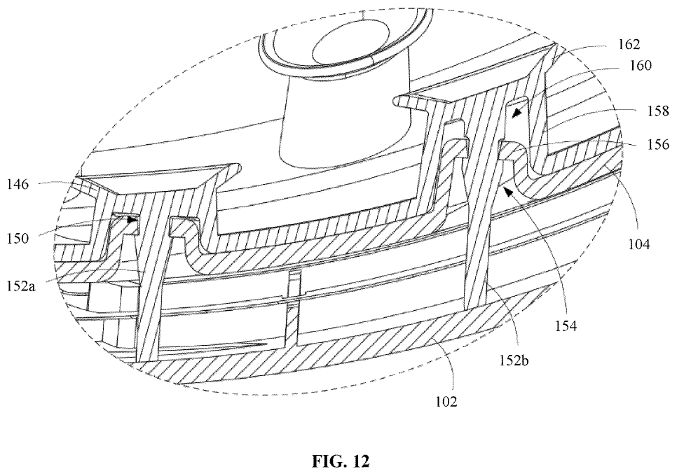

Referring to FIG. 12 illustrates the connection between the plurality of elastic protrusions 146, the inner layer 104, and the outer layer 102. Each plug-in part 152 includes a first plug-in portion 152a and a second plug-in portion 152b. The first plug-in portion 152a is inserted into the second plug-in portion 152b. The first plug-in portion 152a is inserted into the corresponding insertion hole 150 of the inner layer 104, and the second plug-in portion 152b extends into a corresponding recessed portion 154 formed in the inner layer 104. The plurality of elastic protrusions 146 are shown where each includes a sleeve part 158 formed in the inner layer 104. Each of the plurality of elastic protrusions includes the sleeve part 158 and a buffering part 162. The sleeve part 158 enclosed a stimulation element, while the buffering part 162 is in a trumpet shape, and absorbs mechanical shock and provides elasticity. The stimulation element includes but is not limited to a micro-current electrode, a vibration element, a heating element, a cooling element, or a combination thereof. Each elastic protrusion 146 further includes a positioning groove 160 for receiving a positioning protrusion 156 formed on the cavity wall of the inner layer 104, ensuring correct alignment and securing the elastic protrusion 146 in place.

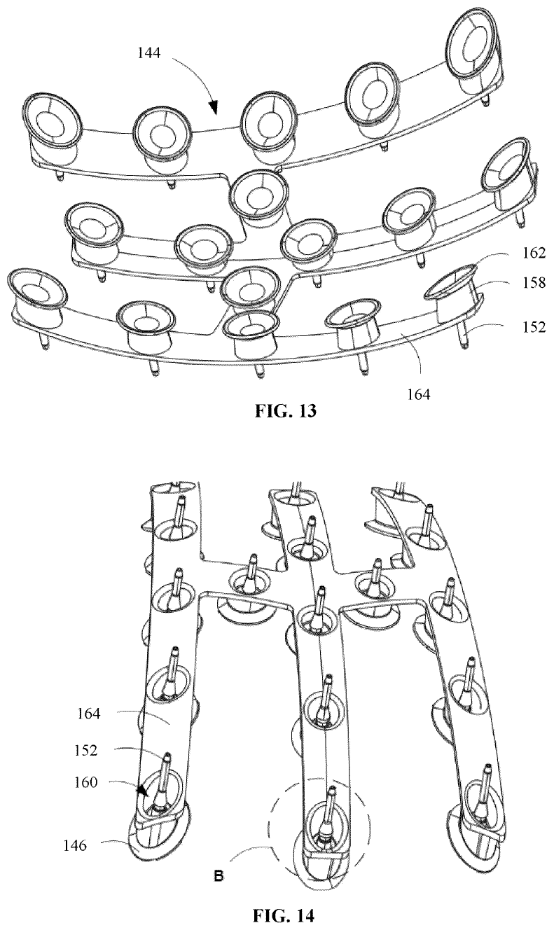

Referring to FIG. 13, the elastic pad 144 comprises the plurality of plug-in parts 152, the plurality of sleeve parts 158, the plurality of buffering parts 162, and a plurality of connecting belts 164. Each plug-in part 152 includes a first plug-in portion and a second plug-in portion and is fixedly coupled to a corresponding elastic protrusion 146. Each elastic protrusion 146 includes the sleeve part 158 and the buffering part 162. The sleeve part 158 extends axially and defines an internal cavity corresponding to the positioning groove 160. The buffering part 162 is generally disposed on one axial end of the sleeve part 158 and is flared to form a trumpet-shaped surface adapted to contact the scalp of a user during use. The plurality of connecting belts 164 are structurally integrated with the elastic pad 144 and couple the plurality of clastic protrusions 146 in an orthogonal matrix, enabling flexible arrangement and mechanical compliance during deformation of the flexible light therapy helmet 100.

Referring to FIG. 14, the plurality of connecting belts 164 forms a grid-like structure, supporting the plurality of plug-in parts 152 and corresponding elastic protrusions 146. The positioning groove 160 defined within the sleeve part 158 is visible for receiving the positioning protrusion 156 formed on the inner layer 104. This mechanical alignment feature ensures secure coupling of the elastic pad 144 within the wearing space 112 of the helmet 100 and facilitates stable positioning of each elastic protrusion 146 during wear. The configuration ensures accurate alignment of the protrusions relative to the user's scalp regions while enabling ventilation and pressure mitigation during therapeutic application.

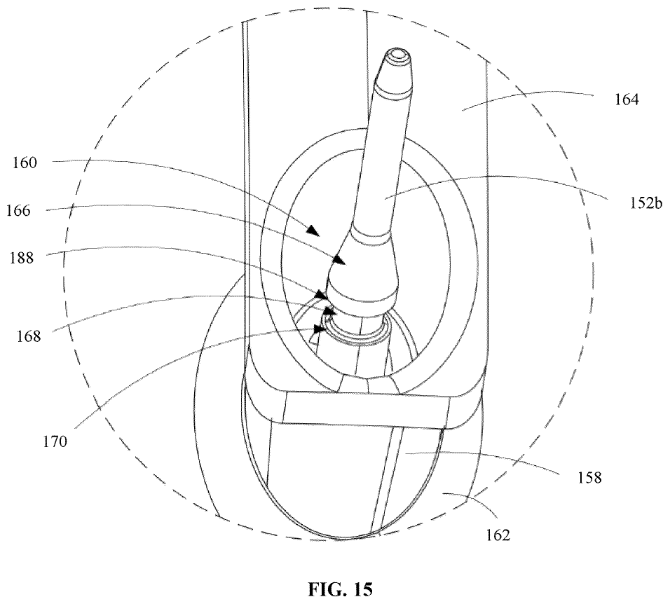

Referring to FIG. 15, the sleeve part 158 surrounds the second plug-in portion 152b, which extends into the inner layer 104 through the corresponding insertion hole 150 and terminates with the positioning groove 160. The internal structure further includes an inclined surface 166 tapering toward an annular groove 168 that is flanked by a first limiting surface 188 and a second limiting surface 170. These elements mechanically engage with corresponding surfaces of the inner layer 104 and the positioning protrusion 156 to prevent axial displacement of the plug-in part 152, ensuring that the stimulation component housed within remains correctly positioned. The buffering part 162, being broader and flexible, contributes to mechanical damping when external pressure is applied.

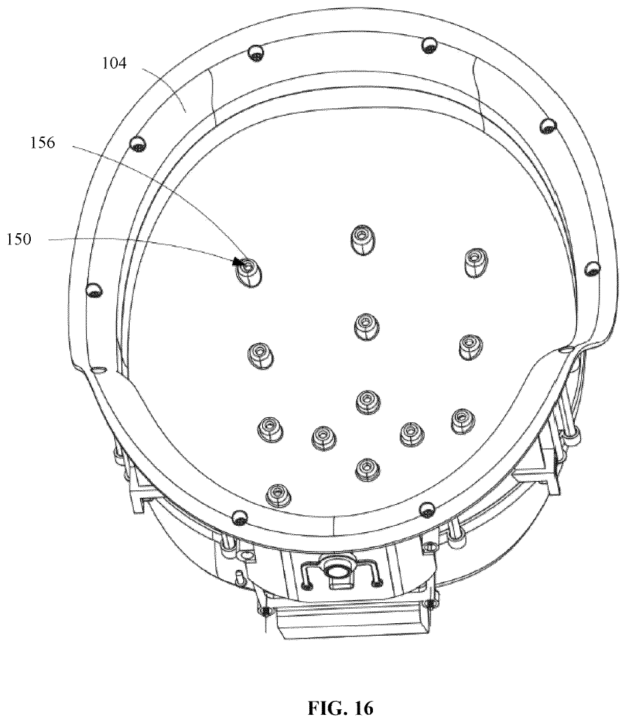

Referring to FIG. 16, the positioning protrusions 156 are distributed across the cavity wall of the inner layer 104 in a geometrically regular manner and are configured to mate with the positioning grooves 160 of the plurality of elastic protrusions 146. Each positioning protrusion 156 enables stable seating and locking of the elastic pad 144, while each insertion hole 150 serves as a receiving aperture for a corresponding second plug-in portion 152b. The interlocked configuration of these features ensures that the plurality of elastic protrusions 146 remains securely aligned and embedded within the inner layer 104.

The flexible light therapy helmet 100 comprises the outer layer 102 and the inner layer 104, which together form a double-layer housing structure. The side of the inner layer 104 that faces away from the outer layer 102 defines the wearing space 112. The cavity wall of the wearing space 112 corresponds to the interior-facing surface of the inner layer 104. The outer layer 102 and the inner layer 104 can be molded together. An installation space is defined between the outer layer 102 and the inner layer 104. The phototherapy lamp 148 is positioned within this space and fixed in place. The inner layer 104 has a light-transmitting region aligned with the phototherapy lamp 148, allowing therapeutic light to be emitted toward the user.

As shown, the plurality of insertion holes 150 are formed through the inner layer 104. Each insertion hole 150 corresponds to the positioning protrusion 156 formed on an interior surface of the inner layer 104 and the corresponding recess portion 154 on the opposite side of the inner layer 104. In an embodiment, the positioning protrusion 156 and the corresponding recess portion 154 are formed by stamping when the inner layer 104 is composed of a metal sheet, or during injection molding of the inner layer 104 in the case of non-metallic inner layer 104.

The plurality of plug-in parts 152 is configured to be fixed within the plurality of insertion holes 150 by adhesive bonding. The corresponding recess portion 154 serves as a bonding cavity to accommodate glue, increasing the structural integrity of the connection. Each plug-in part 152 includes the first limiting surface 188 configured to abut against the inner surface of the inner layer 104. This contact surface prevents axial displacement of the plug-in part 152 into the wearing space 112. In one example, the plug-in part 152 has a stepped cylindrical profile such that the first limiting surface 188 is formed at the interface between a larger-diameter and a smaller-diameter section of the plug-in part 152.

Furthermore, the annular groove 168 is formed around the plug-in part 152. The opposing walls of the annular groove 168 define the first limiting surface 188 and the second limiting surface 170, which bear against opposite sides of the inner layer 104 to restrict axial movement in both directions. This configuration maintains a fixed distance between the buffer portion of the corresponding clastic protrusion 146 and the positioning protrusion 156, ensuring mechanical stability.

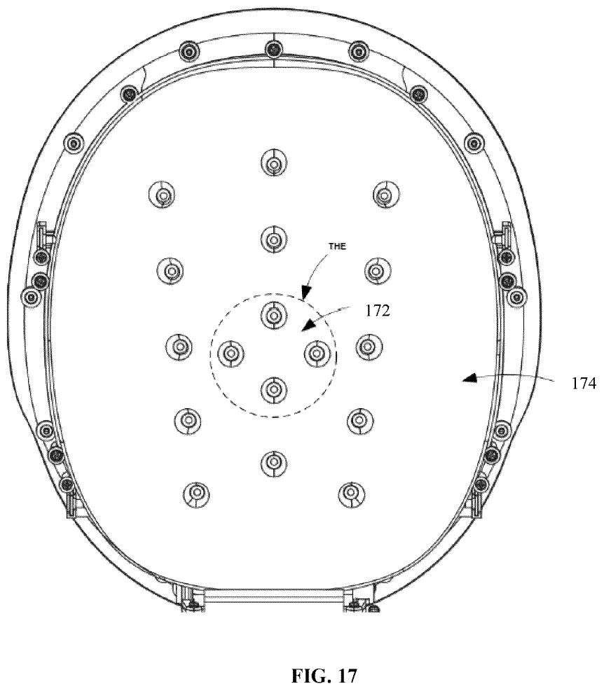

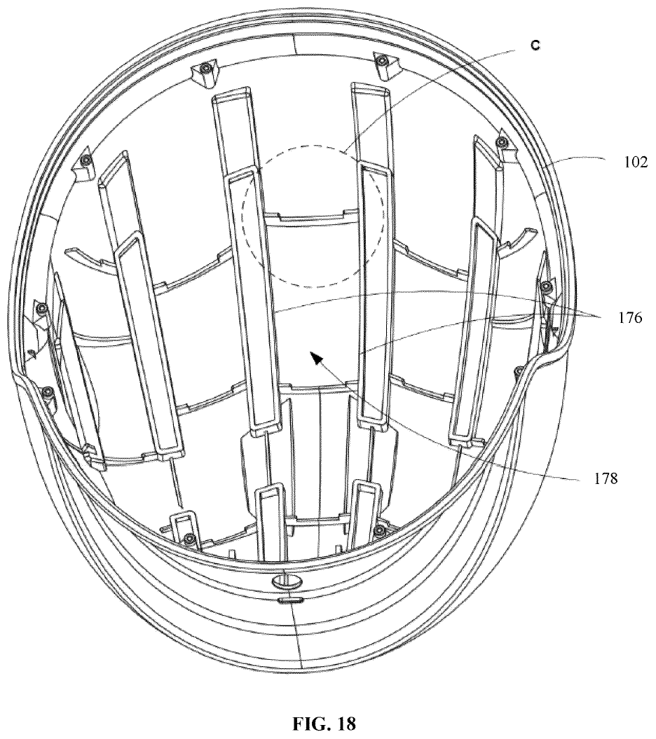

Referring to FIG. 17, the inner layer 104 of the flexible light therapy helmet 100 includes a middle area 172 and an edge area 174. The middle area 172 is centrally located on the inner surface of the inner layer 104 generally corresponding to the region intended to face the top of a user's head when the flexible light therapy helmet 100 is worn. The edge area 174 surrounds the middle area 172 and is positioned circumferentially along the periphery of the inner layer 104. A notional circular boundary delincates the transition between the middle area 172 and the edge area 174.

The wearing space 112 formed by the inner layer 104 and the outer layer 102 exhibits a greater cavity depth in the middle area 172 than in the edge area 174. The plurality of plug-in parts 152 are disposed across the inner layer 104. Each of the plurality of plug-in parts 152 includes the first plug-in portion 152a positioned in the middle area 172 and the second plug-in portion 152b positioned in the edge area 174. Each of the second plug-in portions 152b is provided with the annular groove 168, which includes a first limiting surface 188 and a second limiting surface 170, thereby axially securing the second plug-in portion 152b with respect to the inner layer 104.

In the middle area 172, each elastic protrusion 146 includes a buffering part 162 oriented such that the flared end of the buffering part 162 directly abuts the corresponding positioning protrusion 156 formed on the inner layer 104. This direct contact stable support without requiring significant bending deformation of the buffering part 162, as the compressive force during wear aligned with the axial direction of the elastic protrusion 146.

By contrast, in the edge area 174, the curvature of the inner layer 104 relative to the middle area 172 increases. During donning, this geometry causes the user's head to exert an upward force on the plurality of clastic protrusions 146 in the edge area 174. As a result, a controlled gap is formed between the buffering part 162 and the positioning protrusion 156. This gap allows the buffering part 162 to elastically deform and bend relative to the sleeve part 158, thereby reducing resistance during insertion and improving wearing comfort.

Referring to FIG. 18, a plurality of limiting ribs 176 are disposed longitudinally along the interior surface of the outer layer 102. The plurality of limiting ribs 176 defines corresponding limiting grooves 178 extending parallel to each other in the lengthwise direction. These limiting grooves 178 are configured to receive one or more flexible belts 184 of the PCB 138. Each limiting groove 178 serves to maintain alignment and restrict displacement of the one or more flexible belts 184 within the cavity formed between the outer layer 102 and the inner layer 104.

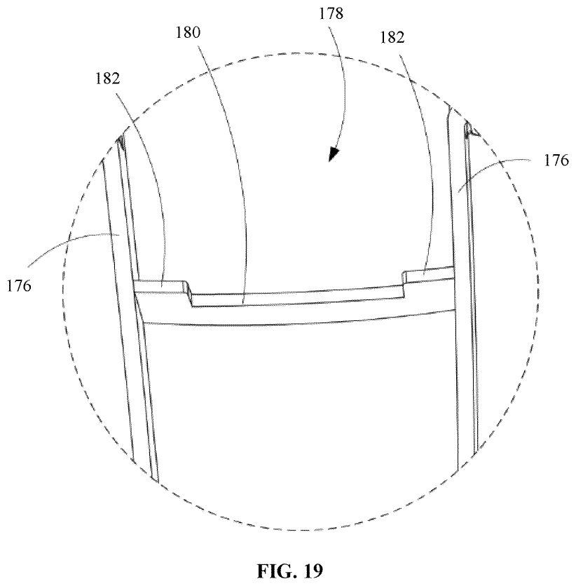

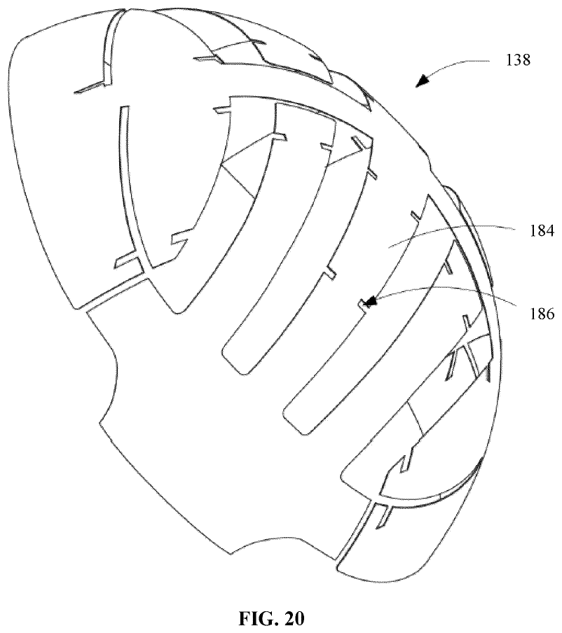

Referring to FIG. 19, within the corresponding limiting groove 178, a support substrate 180 is disposed at the base of the corresponding limiting groove 178. A pair of limiting convex portions 182 is integrally formed on the support substrate 180 and is spaced apart from one another. The one or more flexible belts 184 are placed over the support substrate 180 and positioned between the plurality of limiting convex portions 182. This structural configuration prevents the one or more flexible belts 184 from shifting laterally or longitudinally within the corresponding limiting groove 178 during operation. The support substrate 180 also elevates the one or more flexible belts 184 from the bottom surface of the corresponding limiting groove 178, thereby creating an air gap that facilitates passive heat dissipation of the light-emitting components mounted thereon.

Referring to FIG. 20, a configuration of the PCB 138 is depicted. The PCB 138 comprises the one or more flexible belts 184 extending orthogonally across the longitudinal belts. The one or more flexible belts 184 are interconnected and joined at their ends to form a continuous structure. Each flexible belt 184 includes a plurality of limiting notches 186 formed on opposite sides, which engages with the plurality of limiting convex portions 182 to ensure mechanical retention and optical alignment of the one or more flexible belts 184 within the corresponding limiting groove 178. The PCB 138 is configured to support the LEDs 136 and conforms to the curvature of the outer layer 102 for uniform illumination. The structural assembly of the plurality of limiting ribs 176, limiting grooves 178, the support substrate 180, the plurality of limiting convex portions 182, and the plurality of limiting notches 186 collectively ensure precise and stable placement of the PCB 138.

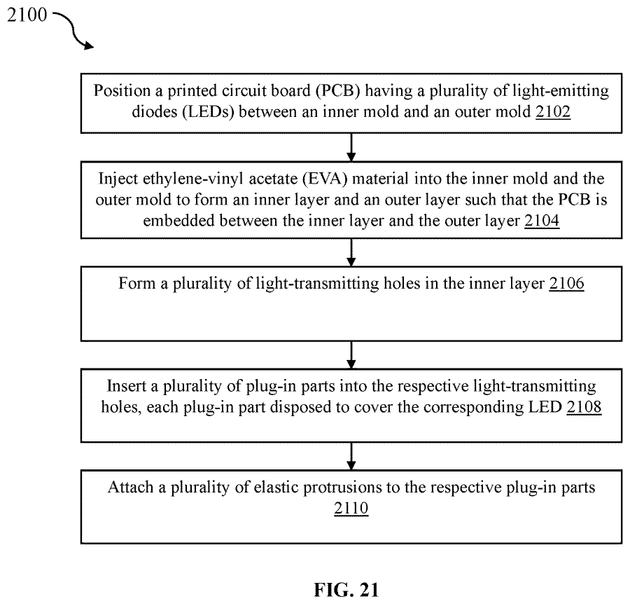

Referring to FIG. 21, a flowchart of method 2100 for manufacturing the flexible light therapy helmet 100 is illustrated, in accordance with an embodiment of the present disclosure. FIG. 21 is explained in conjunction with FIGS. 1-20. The method 2100 may include a plurality of steps for manufacturing the flexible light therapy helmet 100.

At step 2102, a printed circuit board (PCB) 138 is positioned between an inner mold and an outer mold. The PCB 138 includes the plurality of LEDs 136 affixed thereon, which will serve as the phototherapy sources. The placement of the PCB 138 in the mold assembly is critical to ensuring precise alignment and encapsulation during subsequent molding.

Further at step 2104, an ethylene-vinyl acetate (EVA) material is injected into the inner mold and the outer mold to form a layered helmet structure. The EVA material is distributed such that the resulting helmet includes the inner layer 104 and the outer layer 102, with the PCB 138 securely embedded in between. This integral encapsulation provides electrical insulation, thermal regulation, and mechanical protection for the embedded light-emitting circuit.

Further at step 2106, a plurality of light-transmitting holes 114 are formed in the inner layer 104 of the flexible light therapy helmet 100. The plurality of light-transmitting holes 114 are precisely aligned with the positions of the LEDs 136 on the PCB 138. The plurality of light-transmitting holes 114 allows emitted light from the LEDs 136 to pass through the inner layer 104 and reach the user's scalp or treatment area during use.

Further at step 2108, a plurality of plug-in parts 152 are inserted into the corresponding light-transmitting holes 114 in the inner layer 104. Each plug-in part 152 is disposed of in such a manner that it directly covers or encapsulates an underlying LED 136. These plug-in parts 152 may not only help focus and direct the emitted light but also serve as connection points for mechanical attachments.

Further at step 2110, a plurality of elastic protrusions 146 are attached to the respective plug-in parts 152. The plurality of elastic protrusions 146 are configured to extend inward toward the accommodating cavity and contact the user's scalp during wear. These elastic protrusions provide physical cushioning, improve wearing comfort, and maintain appropriate spacing between the light sources and the user's scalp to ensure even light distribution.

Thus, the disclosed method 2100 and the flexible light therapy helmet 100 overcome the challenges of traditional phototherapy devices by providing a compact, integrally molded, and user-conforming structure that improves manufacturing efficiency, comfort, and therapeutic effectiveness. The method allows for embedding a printed circuit board (PCB) between an inner layer and an outer layer using ethylene-vinyl acetate (EVA), thereby ensuring electrical insulation, mechanical protection, and a lightweight construction.

Additionally, by forming a plurality of light-transmitting holes aligned with LEDS and integrating plug-in parts with elastic protrusions, the flexible light therapy helmet 100 ensures uniform light delivery to the scalp without direct contact between the LEDs and skin. This prevents thermal discomfort, enables airflow through ventilation paths, and maintains safe spacing for effective irradiation. Moreover, the use of cushion blocks allows for better fitting across varying head shapes and improves user experience during prolonged wear. Overall, the design enhances breathability, light penetration, and mechanical stability, addressing deficiencies in wearability, assembly complexity, and phototherapy consistency in existing helmet-based devices.

As will be appreciated by those skilled in the art, the design described in the various embodiments discussed above is not routine, conventional, or well-understood in the art. In light of the above-mentioned advantages and the technical advancements provided by the disclosed method and the multifunctional comb, the claimed steps as discussed above are not routine, conventional, or well understood in the art, as the claimed steps enable the following solutions to the existing problems in conventional technologies. Further, the claimed steps bring an improvement in the functioning of the device itself as the claimed steps provide a technical solution to a technical problem.

The specification has described a flexible light therapy helmet and a method of manufacturing the same. The illustrated steps are set out to explain the exemplary embodiments shown, and it should be anticipated that ongoing technological development will change the manner in which particular functions are performed. These examples are presented herein for the purpose of illustration and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

It is intended that the disclosure and examples be considered as exemplary only, with a true scope of disclosed embodiments being indicated by the following claims.

1. A flexible light therapy helmet comprising:
an outer layer formed of Ethylene Vinyl Acetate (EVA) material,
wherein the outer layer defines a wearing space;
an inner layer formed of the EVA material,
wherein the inner layer is disposed on a side of the outer layer facing the wearing space;
a printed circuit board (PCB) disposed between the inner layer and the outer layer, wherein the PCB comprising a plurality of light-emitting diodes (LEDs); and
a plurality of light-transmitting holes formed in the inner layer, wherein each light-transmitting hole is aligned with a corresponding LED to transmit therapeutic light toward a user's scalp.

2. The flexible light therapy helmet of claim 1, wherein the inner layer and the outer layer are fixed together via glue or via the injection molding process.

3. The flexible light therapy helmet of claim 1, wherein the inner layer and the outer layer are formed by an injection molding process.

4. The flexible light therapy helmet of claim 2, wherein each light-transmitting hole comprises a plug-in part covering the LED and extending outward from the inner layer, and wherein the plug-in part comprises a lens configured to enhance the light emitted by the LED.

5. The flexible light therapy helmet of claim 3, wherein the plug-in part is coupled to an elastic pad, and wherein the elastic pad comprising an elastic belt and a plug-in receiving hole for receiving the corresponding plug-in part.

6. The phototherapy helmet of claim 4, wherein the plug-in part is integrally formed with the elastic pad, and the elastic pad is affixed to the inner layer.

7. The phototherapy helmet of claim 4, wherein the elastic pad comprises a conductive pathway to supply electrical power to a stimulation element.

8. The phototherapy helmet of claim 1, wherein the inner layer and the outer layer comprise one or more air vents.

9. A flexible light therapy helmet comprising:
an outer layer formed of Ethylene Vinyl Acetate (EVA) material via an injection molding process,
wherein the outer layer defines a wearing space;
an inner layer formed of the EVA material,
wherein the inner layer is disposed on a side of the outer layer facing the wearing space, and wherein the inner layer is joined with the outer layer;
a printer circuit board (PCB) disposed between the inner layer and the outer layer,
wherein the PCB comprises a plurality of light-emitting diodes (LEDs);
a plurality of light-transmitting holes formed in the inner layer, wherein each light-transmitting hole is aligned with a corresponding LED to transmit therapeutic light toward a user's scalp;
a plurality of plug-in parts each disposed within a corresponding light-transmitting hole; and
a plurality of elastic protrusions coupled to the plug-in parts, each elastic protrusion comprising a secondary stimulation element configured to deliver one or more electrical, vibrational, or thermal stimulation to a user's scalp.

10. The flexible light therapy helmet of claim 9, wherein the secondary stimulation element is selected from a group consisting of: an electrode, a vibration motor, and a thermal module.

11. The flexible light therapy helmet of claim 9, wherein each elastic protrusion comprises a silicon tip and a metal contact surface configured to contact the user's scalp.

12. The flexible light therapy helmet of claim 9, wherein the plug-in part comprises an annular groove having a first limiting surface and a second limiting surface configured to abut opposite sides of the inner layer.

13. The flexible light therapy helmet of claim 9, wherein the elastic protrusion comprises a sleeve portion housing the stimulation element and a buffer portion to absorb mechanical pressure.

14. The flexible light therapy helmet of claim 9, wherein the plug-in parts and elastic protrusions are integrally formed with the elastic pad.

15. The flexible light therapy helmet of claim 9, further comprising a decorative strip disposed on an external surface of the outer layer.

16. The flexible light therapy helmet of claim 9, wherein the PCB is mounted on a flexible substrate and comprises arc-shaped light strips arranged in longitudinal and transverse directions.

17. A method for manufacturing a flexible light therapy helmet, the method comprising:
positioning a printed circuit board (PCB) having a plurality of light-emitting diodes (LEDs) between an inner mold and an outer mold;
injecting ethylene-vinyl acetate (EVA) material into the inner mold and the outer mold to form an inner layer and an outer layer such that the PCB is embedded between the inner layer and the outer layer; and
forming a plurality of light-transmitting holes in the inner layer, wherein each light-transmitting hole is aligned with a corresponding LED.

18. The method of claim 17, further comprising:
inserting a plurality of plug-in parts into the respective light-transmitting holes, each plug-in part disposed to cover the corresponding LED.

19. The method of claim 18, further comprising:
attaching a plurality of elastic protrusions to the respective plug-in parts, wherein each elastic protrusion comprising a stimulation element configured to provide one or more of electrical, vibrational, or thermal stimulation to a user.

20. The method of claim 17, wherein forming the plurality of light-transmitting holes comprises positioning a plurality of core pins in the mold aligned with the LEDs before injection of the EVA material.
